(19) United States
(12) Patent Application Publication
(10) Pub. No.: US 2025/0121157 A1
(43) Pub. Date: Apr. 17, 2025

(54) MEDICAL TUBE CLEARANCE DEVICE

(71) Applicant: CLEARFLOW, INC., Irvine, CA (US)

(72) Inventors: KATHRYN BERNADINE O'KEEFE, BEND, OR (US); STEPHEN RIDDLE MCDANIEL, SAN FRANCISCO, CA (US); EDWARD M. BOYLE, JR., BEND, OR (US); KENNETH ALLAN BERES, STUDIO CITY, CA (US); MATTHEW CHRISTOPHER BARRA, LEOMINSTER, MA (US); THOMAS URBANIK, SOMERVILLE, MA (US); CHELSEA ANN GAMMON, SAN DIEGO, CA (US); KENNETH J. CHESNIN, LONG BEACH, CA (US)

(21) Appl. No.: 18/989,536

(22) Filed: Dec. 20, 2024

Related U.S. Application Data
(63) Continuation of application No. 16/593,547, filed on Oct. 4, 2019, now Pat. No. 12,171,952.
Application No. 16/593,547 is a continuation of application No. 14/624,161, filed on Feb. 17, 2015, now Pat. No. 10,471,189.
(60) Provisional application No. 61/940,713, filed on Feb. 17, 2014.

(51) Int. Cl. A61M 25/00; A61M 1/00

(52) U.S. Cl. CPC A61M 25/00; A61M 1/83; A61M 1/84; A61M 2025/0008; A61M 2025/0019; A61M 2209/10

(57) ABSTRACT

A medical device for clearing obstructions from a medical tube includes an enclosure defining an interior, an exterior, and an opening for providing access to the interior. The device further includes a drive mechanism and an elongated guide member. The drive mechanism is configured to advance the elongated guide member out of the enclosure through the opening without compromising a sterile field and withdraw the elongated guide member into the enclosure through the opening without compromising the sterile field.

### CROSS-REFERENCE TO RELATED APPLICATIONS

This application claims the benefit of U.S. patent application Ser. No. 16/593,547, filed Oct. 4, 2019, which claims the benefit of U.S. patent application Ser. No. 14/624,161, filed Feb. 17, 2015, which claims the benefit of U.S. Provisional Application No. 61/940,713, filed Feb. 17, 2014, which are incorporated in their entireties herein by reference.

### TECHNICAL FIELD

This application relates generally to a medical tube assembly and, more specifically, to a device for clearing obstructions from a medical tube of the medical tube assembly.

### BACKGROUND

Medical tubes can be used to deliver fluids or devices into a body and/or drain bodily fluids and secretions from compartments and structures within the body. For example, medical tubes can be used to drain fluid from one's bladder, from the colon or other portions of the alimentary tract, or from the lungs or other organs in conjunction with various therapies. As another example, medical tubes can be used to drain blood and other fluids that typically accumulate within the body cavity following traumatic surgery. As yet another example, medical tubes can be used to deliver fluids to a body for nourishment or they can be used to provide access to the vasculature for removal or delivery of fluids or devices. Typically, a medical tube is inserted into the patient so that its distal end is provided in or adjacent the space where it is desired to remove or deliver material while a proximal portion remains outside the patient's body, where it can be connected, for example, to a suction source.

Fluids passing through a medical tube (particularly those including blood or blood platelets) can form clots or other obstructions within the medical tube, which can partially or totally obstruct the suction pathway within the tube. Obstruction of the medical tube can impact its effectiveness to remove or deliver the fluid and other material for which it was originally placed, eventually rendering the medical tube partially or totally non-functional. In some cases, a non-functional tube can have serious or potentially life-threatening consequences. For example, if there is a blockage in a chest tube following cardiac or pulmonary surgery, the resulting accumulation of fluid around the heart and lungs without adequate drainage can cause serious adverse events such as pericardial tamponade and pneumothorax.

### SUMMARY

The following presents a simplified summary of the disclosure in order to provide a basic understanding of some example aspects described in the detailed description.

In accordance with a first aspect, a medical device is provided for clearing obstructions from a medical tube. The medical device includes an enclosure defining (i) an interior comprising a sterile field, a chamber, and a discrete conduit in communication with and extending substantially tangentially of said chamber, (ii) an exterior, and (iii) an opening for providing access to the interior. The medical device further includes a drive mechanism and an elongated guide member. The drive mechanism is configured to (i) advance the elongated guide member out of the enclosure through the opening without compromising the sterile field and (ii) withdraw the elongated guide member into the enclosure through the opening without compromising the sterile field.

### BRIEF DESCRIPTION OF THE DRAWINGS

Embodiments of the invention are better understood when the following detailed description is read with reference to the accompanying drawings, in which:

FIG. 1 is a schematic view of a medical tube assembly;

FIG. 2 is an exploded view of a device for clearing obstructions from a medical tube of the medical tube assembly;

FIG. 3 is a perspective view of an example guide member and clearance member of the device;

FIG. 4 is a perspective view of another example guide member and clearance member of the device;

FIG. 5 is a perspective view of the guide member and clearance member shown in FIG. 4 in a reverse configuration;

FIG. 6 is a cross-sectional view of the device coupled to the medical tube with an example medical tube connector;

FIG. 7 is a cross-sectional view of the device coupled to a discharge tube with an example discharge connector;

FIG. 8 is a schematic view of the device coupled to the medical tube with an example y-connector according to a first configuration;

FIG. 9 is a schematic view of the device coupled to the medical tube with the example y-connector according to a second configuration;

FIG. 10 is a cross-sectional view of a drive mechanism of the device according to one example configuration;

FIG. 11 is a cross-sectional view of the drive mechanism of the device according to another example configuration;

FIG. 12 is a schematic view of the device comprising a motor, a transmission mechanism, and a control system;

FIG. 13 is a perspective view of the device comprising a rotating knob;

FIG. 14 is a cross-sectional of the device comprising one or more spool elements and drive elements;

FIG. 15 is a partial broken-away view of the device showing a guide feature of the device according to one example configuration;

FIG. 16 is a similar view as in FIG. 15 showing the guide feature of the device according to another example configuration;

FIG. 17 is a close-up longitudinal cross-sectional view of the device showing a compression element of the device according to one example configuration;

FIG. 18 is a close-up lateral cross-sectional view of the compression element shown in FIG. 17 taken along line 18-18;

FIG. 19 is a close-up longitudinal cross-sectional view of the device showing a compression element of the device according to another example configuration;

FIG. 20 is a close-up lateral cross-sectional view of the compression element shown in FIG. 19 taken along line 20-20;

FIG. 21 is a perspective view of one embodiment of the medical tube assembly that comprises a drainage receptacle;

FIG. 22 is a schematic view of the medical tube according to one example embodiment;

FIG. 23 is a schematic view of the medical tube shown in FIG. 22 after being cut;

FIG. 24 is a schematic view of the medical tube according to another example embodiment;

FIG. 25 is a schematic view of the medical tube shown in FIG. 24 after being cut;

FIG. 26 is a flow chart illustrating steps of a method of calibrating a medical tube; and

FIG. 27 is a flow chart illustrating steps of a method of clearing obstructions from a medical tube using a device for clearing obstructions from the medical tube.

### DETAILED DESCRIPTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is to be noted that the terms “proximal” and “distal” as used herein when describing two ends or portions of a feature indicate a relative positioning that those two ends or portions will generally have along an in-line system that is tied to a patient, the distal end or portion being closer to the patient than the proximal end or portion. For example, in an in-line system comprising a widget that draws fluid from the patient through the widget along a flow path, a distal end or portion of a widget will be closer to a patient than a proximal end or portion of the widget along the flow path of the fluid.

Examples will now be described more fully hereinafter with reference to the accompanying drawings in which example embodiments are shown. Whenever possible, the same reference numerals are used throughout the drawings to refer to the same or like parts. However, aspects may be embodied in many different forms and should not be construed as limited to the embodiments set forth herein.

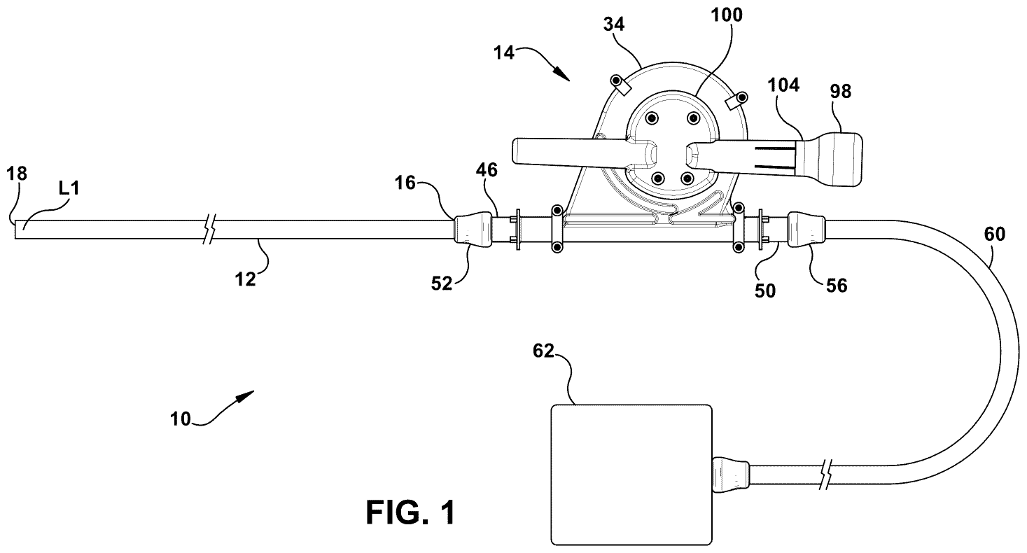

As shown in FIG. 1, a medical tube assembly 10 can comprise a medical tube 12 and a device 14 for clearing obstructions from a medical tube 12. The medical tube 12 can be a tube having a length, an inner diameter, and an outer diameter that can each vary between different embodiments. Indeed, the medical tube 12 can have a variety of different shapes and configurations without departing from the scope of the invention. The medical tube 12 can be used to drain bodily fluids and secretions from within body compartments and structures such as, for example, fluid from within a person's bladder, colon, lungs, brain, thoracic cavity, or any other body structure. The medical tube 12 can alternatively be used to deliver fluids or devices to a body compartment or structure. In some examples, the medical tube 12 can be used to drain bodily fluids and secretions from within body compartments and structures and deliver the fluids to other body compartments and structures.

The medical tube 12 can comprise a proximal opening 16 and a distal opening 18 and can be inserted into a patient so that its distal opening 18 is provided in or adjacent the space where it is desired to remove or deliver material while the proximal opening 16 remains outside the patient's body. In the example shown in FIG. 1, the proximal opening 16 and distal opening 18 respectively coincide with a proximal and distal end of the medical tube 12. However, in some examples, the proximal opening 16 and/or distal opening 18 may be openings along the medical tube 12 that are intermediate its ends.

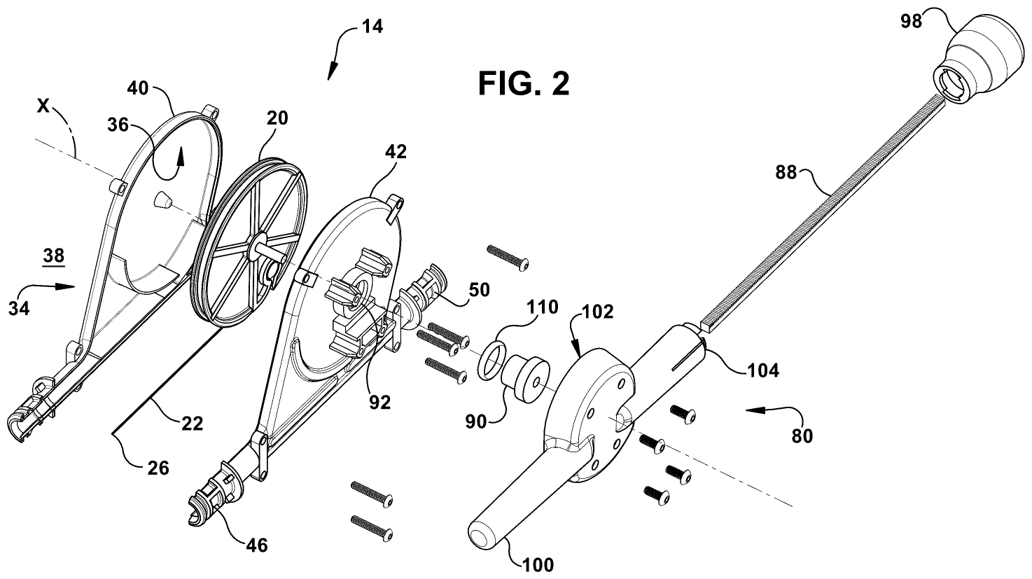

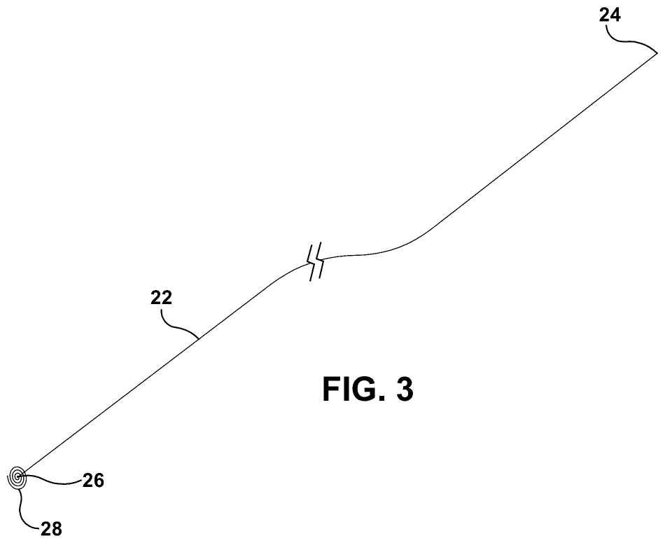

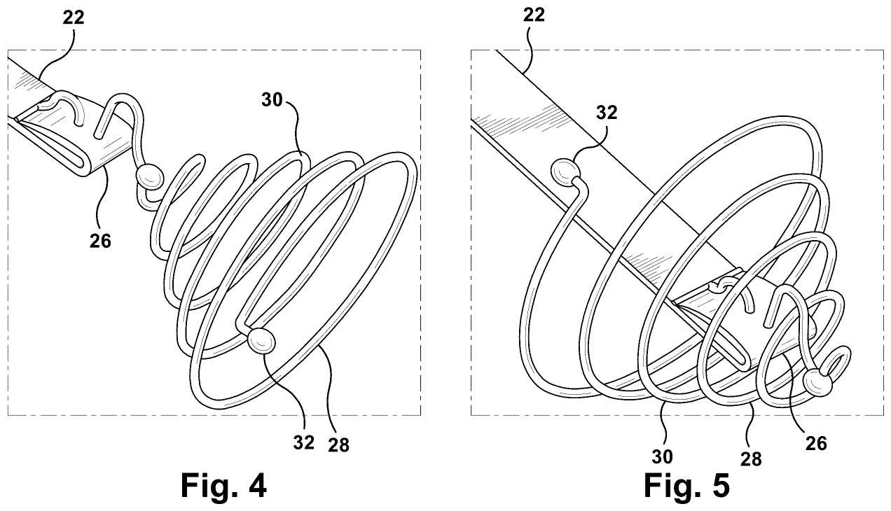

Turning to FIGS. 2-5, the device 14 can comprise a spool 20 that is rotatable about an axis X and an elongated guide member 22 which, as will be discussed in further detail below, can be advanced or withdrawn through the medical tube 12 to help dislodge and/or draw obstructing material within the medical tube 12 without compromising a sterile field within the medical tube 12.

In some examples, the guide member 22 can comprise a wire having a substantially circular cross-section (as shown in FIGS. 2 & 3) while in other examples, the guide member 22 can comprise a flat tape having a substantially rectangular cross-section (as shown in FIGS. 4 & 5). The guide member 22 can comprise a proximal end 24 and a distal end 26. The proximal end 24 of the guide member 22 can be coupled to the spool 20. Moreover, in some embodiments, the device 14 can comprise one or more clearance members 28 that can be coupled to the distal end 26 or other portions of the guide member 22. It is to be noted that the term “coupled” as used herein when describing two or more features means that the features can be integral with each other or that the features can be separate features that are removably or non-removably attached to each other using various means such as threads, fasteners, hooks, clips, adhesive, welds, or other means of attaching two separate features. For example, in some embodiments, the coupled guide member 22 and clearance member 28 may be integral components formed together of a single piece of wire, while the coupled guide member 22 and spool 20 may be separately attached features.

When the guide member 22 is coupled to the clearance member 28 and inserted within the medical tube 12, the guide member 22 can guide the clearance member 28 through the medical tube 12 as the guide member 22 is advanced or withdrawn through the medical tube 12. The clearance member 28 can be configured such that as the clearance member 28 is guided through the medical tube 12 by the guide member 22, the clearance member 28 can help dislodge and/or draw obstructing material within the medical tube 12. For example, the clearance member 28 can comprise a wire 30 comprising a plurality of coils arranged in a spiral configuration, as shown in FIGS. 4 & 5. The wire 30 can comprise a material with elastic or shape memory properties such as, for example, nickel-titanium that allows the plurality of coils to expand or conform to various shapes and sizes of the medical tube 12. Moreover, depending on which direction the clearance member 28 is being translated through the medical tube 12, the plurality of coils can reverse their conformation to produce substantially conical configurations of coils facing opposite directions, thereby reducing the resistance exerted on the clearance member 28 by the walls of the medical tube 12. If the medical tube 12 comprises any fluid intake apertures in its side walls, the wire 30 can comprise an end portion 32 that is sized or otherwise configured so that the end portion 32 will not fit through such apertures and potentially extend laterally out of the medical tube 12.

In other examples, the wire 30 of the clearance member 28 may comprise a loop or other structure that presents substantially no impediment to flow through the medical tube 12 past the clearance member 28 regardless of whether the clearance member 28 is at rest or being actuated within the medical tube 12. In a further embodiment, the clearance member 28 can comprise a brush with a plurality of bristles rather than the wire 30. The clearance member 28 can comprise any member that is translatable through the medical tube 12 to dislodge and/or draw obstructing material accumulated within the medical tube 12.

The guide member 22 can be coupled to the spool 20 such that rotation of the spool 20 about the axis X causes the guide member 22 to wind or unwind about the spool 20 and move between an advanced state and a retracted state. In some embodiments, the distal end 26 of the guide member 22 may be positioned within the medical tube 12 and coupled to the clearance member 28. If the spool 20 is rotated in one direction, the guide member 22 will wind about the spool 20, causing the guide member 22 and the coupled clearance member 28 to translate away from the distal opening 18 of the medical tube 12 (i.e., retract). Alternatively, if the spool 20 is rotated in the opposite direction, the guide member 22 will unwind about the spool 20, causing the guide member 22 and the coupled clearance member 28 to translate toward the distal opening 18 of the medical tube 12 (i.e., advance). Thus, rotation of the spool 20 can control the position and operation (actuation) of the guide member 22 and coupled clearance member 28 within the medical tube 12.

The guide member 22 can comprise a material with elastic or shape memory properties such as, for example, nickel-titanium that allows the guide member 22 to conform to the curvature of the spool 20 when wound about the spool 20 and also allows the guide member 22 take on or resume a more linear shape or configuration when advancing through the medical tube 12. Preferably, the guide member 22 comprises a material that is rigid enough to advance the clearance member 28 through the medical tube 12 when coupled to thereto. However, the guide member 22 may comprise a variety of different shapes, sizes and materials without departing from the scope of the invention.

In some embodiments, one or both of the clearance member 28 and the guide member 22 can be coated with at least one of a pharmacologic material, an anti-thrombogenic material, and an anti-infective material to assist in treating materials inside the medical tube 12.

As shown in FIGS. 2, the device 14 can comprise an enclosure 34 having an interior 36 and an exterior 38. The spool 20 and at least a portion of the guide member 22 can be provided within the interior 36 of the enclosure 34. The device 14 can be configured such that the enclosure 34 can be coupled with the medical tube 12 and the spool 20 can be rotated to advance or retract the guide member 22 within the medical tube 12 without compromising a sterile field within the medical tube 12 and the interior 36 of the enclosure 34.

More specifically, the enclosure 34 can comprise a first half portion 40 and a second half portion 42 that are coupled together. For example, the first and second half portions 40, 42 can be separate portions that are attached to each other using threaded fasteners or other attaching means to form a hermetical seal therebetween. The enclosure 34 can further comprise one or more opening portions for providing access to the interior 36 of the enclosure 34. For example, the enclosure 34 can comprise a distal opening 46 and in some examples, the enclosure 34 can also comprise a proximal opening 50 for providing access to the interior 36 of the enclosure 34.

In some examples, the device 14 can comprise one or more connectors to couple the opening portions of the enclosure 34 to the medical tube 12 or other structure and form a closed passageway therebetween. It to be noted that the phrase “closed passageway” as used herein is meant to describe a passageway that is not exposed to an exterior environment between its inlet and outlet, thereby preserving a sterile field that may be present within the passageway. For instance, as shown in FIGS. 1 & 6, the device 14 can comprise a medical tube connector 52. The medical tube connector 52 can be coupled to the distal opening 46 of the enclosure 34 and is removably coupleable to the medical tube 12 to form a first closed passageway 54 for fluid communication between the enclosure 34 and the medical tube 12 through the medical tube connector 52. The device 14 can also comprise a drainage connector 56, as shown in FIGS. 1 & 7. The drainage connector 56 can be coupled to the proximal opening 50 of the enclosure 34 and is removably coupleable to drainage structure such as, for example, a drainage tube 60 of the medical tube assembly 10, to form a second closed passageway 64 for fluid communication between the enclosure 34 and the drainage structure through the proximal opening 50 of the enclosure 34. As such, the medical tube 12, drainage tube 60, and enclosure 34 can form a closed passageway wherein the medical tube 12 and the drainage tube 60 are in fluid communication with each other through the enclosure 34. The medical tube assembly 10 can comprise a vacuum source 62 that can be coupled to a proximal end of the drainage tube 60 to selectively provide a vacuum that draws fluid from a patient into the medical tube 12, then from the medical tube 12 into the enclosure 34 through the distal opening 46 of the enclosure 34, and then from the enclosure 34 into the drainage tube 60 through the proximal opening 50 of the enclosure 34.

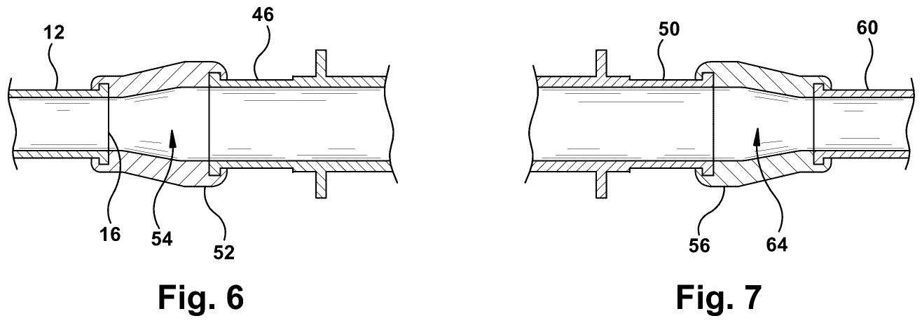

As can be seen in FIG. 6, the medical tube connector 52 can have a first internal diameter that is in continuity with an internal diameter of the medical tube 12 and a second internal diameter in continuity with an internal diameter of the distal opening 46 of the enclosure 34, thus providing the first closed passageway 54 with a continuously smooth pathway between the medical tube 12 and the distal opening 46. Similarly, as can be seen in FIG. 7, the drainage connector 56 can be a straight connector that has a variable internal diameter (similar as the medical tube connector 52 just described) that is in continuity with both an internal diameter of the drainage tube 60 and an internal diameter of the proximal opening 50 of the enclosure 34, thus providing the second closed passageway 64 with a continuously smooth pathway between the drainage tube 60 and the proximal opening 50.

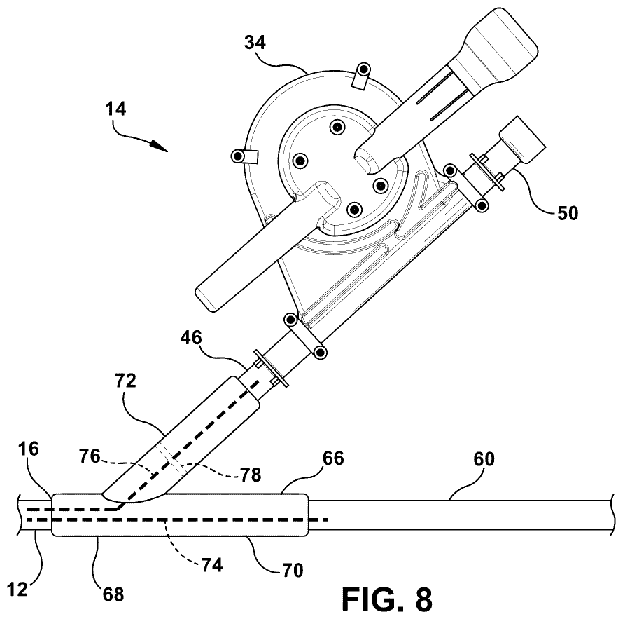

In some embodiments, the device 14 can comprise a 3-way connecter 66 that is coupled to the distal opening 46 of the enclosure 34 and removably coupleable to the medical tube 12 and optionally the drainage tube 60, as shown in FIGS. 8 & 9. For example, the 3-way connecter 66 can comprise a primary branch 68 that splits into an axial branch 70 and a lateral branch 72. The axial branch 70 can be arranged substantially linear and coaxial with the primary branch 68 to form a linear pathway 74 and the lateral branch 72 can be arranged so that it extends in a lateral direction from the primary branch 68 to form an angled pathway 76.

The primary branch 68 of the 3-way connecter 66 can be coupled to the medical tube 12. Meanwhile, the axial and lateral branches 70, 72 can be capped or coupled to either the distal opening 46 of the enclosure 34 or drainage structure such as the drainage tube 60. For example, in one embodiment, the axial branch 70 may be coupled to the drainage tube 60 and the lateral branch 72 may be coupled with the distal opening 46 of the enclosure 34, as shown in FIG. 8. If the enclosure 34 comprises the proximal opening 50, the proximal opening 50 can be capped to close access to the enclosure 34 from the external environment through the proximal opening 50. The medical tube 12, drainage tube 60, and enclosure 34 can thus form a closed passageway wherein the medical tube 12 and the drainage tube 60 are in fluid communication with each other through the linear pathway 74. The vacuum source 62 can be coupled to the drainage tube 60 to selectively provide a vacuum that draws fluid from a patient into the medical tube 12, through the linear pathway 74, and then into the drainage tube 60 without passing through the enclosure 34.

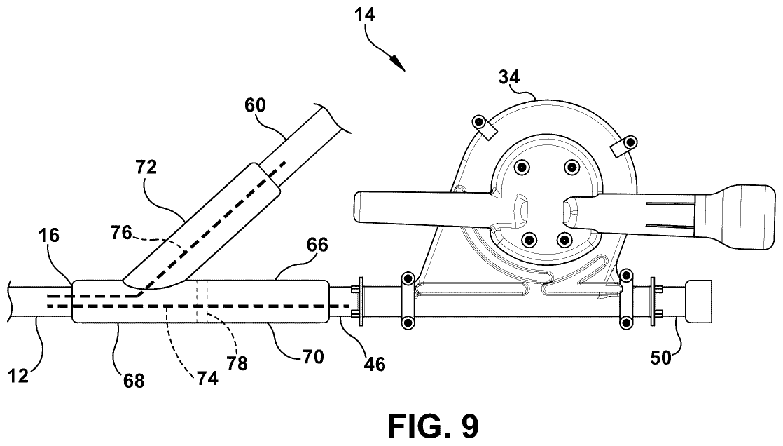

In another embodiment, the axial branch 70 can be coupled to the distal opening 46 of the enclosure 34 and the lateral branch 72 can be coupled to the drainage tube 60, as shown in FIG. 9. If the enclosure 34 comprises the proximal opening 50, the proximal opening 50 can be capped. The medical tube 12, drainage tube 60, and enclosure 34 can thus form a closed passageway wherein the medical tube 12 and the drainage tube 60 are in fluid communication with each other through the angled pathway 76. The vacuum source 62 can be coupled to the drainage tube 60 to selectively provide a vacuum that draws fluid from a patient into the medical tube 12, through the angled pathway 76, and then into the drainage tube 60 without passing through the enclosure 34.

The connectors 52, 56, 66 described above can be rigid or flexible structures that can be coupled to the medical tube 12, enclosure 34, drainage tube 60, or other drainage structure to provide a variety of different closed passageways. Moreover, the connectors 52, 56, 66 can be coupled to the medical tube 12, enclosure 34, drainage tube 60, or other drainage structure using various means such as threaded couplings, quarter-turn locks, push-button locks, or other quick-connect means that will preserve a sterile field within and between the connected elements. Furthermore, in some embodiments, the tube 12, enclosure 34, drainage tube 60, or other drainage structure can be coupled together using other connecters such as, for example, T-connecters or connecters with more than three ports.

In some examples, the device 14 can comprise an isolation member 78 that can be configured to help isolate the enclosure 34 from the medical tube 12 when the device 14 is coupled to the medical tube 12 using a connecter such as, for example, one of the connecters 52, 66 described above. For instance, when the device 14 is coupled to the medical tube 12 as shown in FIG. 8, the isolation member 78 may be provided within the lateral branch 72 of the 3-way connecter 66 to inhibit fluid communication through the lateral branch 72 but permit the guide member 22 to translate through, thus helping to isolate the enclosure 34 from the fluid and debris traveling through the linear pathway 74. As another example, when the device 14 is coupled to the medical tube 12 as shown in FIG. 9, the isolation member 78 can be provided within the axial branch 70 of the 3-way connecter 66 to inhibit fluid communication through the axial branch 70 but permit the guide member 22 to translate through, thus helping to isolate the enclosure 34 from the fluid and debris traveling through the angled pathway 76. In other examples, the isolation member 78 may be provided within the connecter 52. Indeed, in some examples, the isolation member 78 may be located in-line between the enclosure 34 and a connecter or between the medical tube 12 and a connecter. The isolation member 78 can be a seal and/or valve and may be contained within any housing.

When the device 14 is coupled to the medical tube 12 using, for example, the connecters 52, 66 described above, a closed passageway can be formed between the enclosure 34 and the medical tube 12 for the guide member 22 to extend through. More specifically, a distal portion of the guide member 22 can extend through the distal opening 46 of the enclosure 34 and into the medical tube 12. A remaining, proximal portion of the guide member 22 can remain within the enclosure 34 and coupled to the spool 20. As the guide member 22 is wound onto or off of the spool 20, the guide member 22 will be respectively retracted or advanced through the distal opening 46 of the enclosure 34 and the medical tube 12.

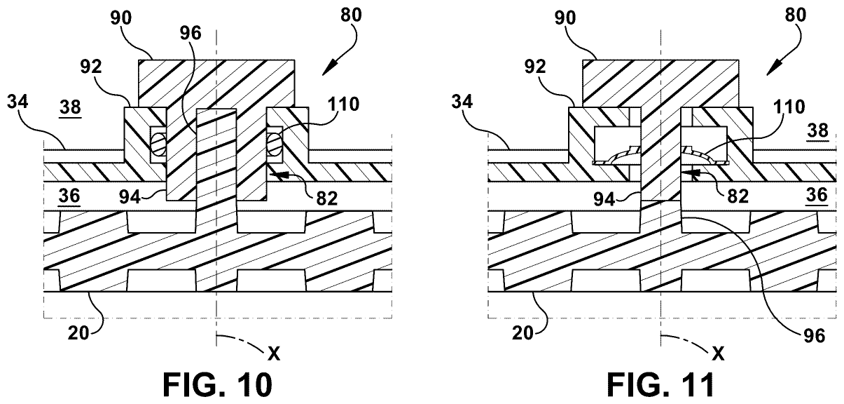

The device 14 can further comprise a drive mechanism 80 that is operable to rotate the spool 20 within the enclosure 34 and move the guide member 22 between an advanced state and a retracted state without compromising a sterile field within the enclosure 34; i.e. without exposing the interior of the enclosure 34 to the exterior environment. In a first example embodiment the drive mechanism 80 can comprise a drive shaft 82, a rack 88, and a pinion gear 90, as shown in FIGS. 2 & 10-11. The drive shaft 82 can extend through a drive shaft opening 92 of the enclosure 34. Moreover, the drive shaft 82 can be coaxial with and coupled to the spool 20 and in some examples the drive shaft 82 can be coaxial with and coupled to the pinion gear 90. For instance, as shown in FIG. 10, the drive shaft 82 can be an assembly comprising a stem portion 94 of the pinion gear 90 and an axle portion 96 of the spool 20 that is inserted within the stem portion 94 of the pinion gear 90 and coupled thereto. However, in some examples, the stem portion 94 of the pinion gear 90 may be inserted within the axle portion 96 of the spool 20 and coupled thereto. Moreover, in other examples, the drive shaft 82 may comprise just the stem portion 94 of the pinion gear 90 (as shown in FIG. 11), just the axle portion 96, or some other structure/assembly that extends through the drive shaft opening 92 and is coaxial with and coupled to the spool 20.

As shown in FIG. 2, the rack 88 can be provided outside of the enclosure 34 and can be engaged with the pinion gear 90 such that linear translation of the rack 88 causes the pinion gear 90 and thereby spool 20 to rotate about the axis X. In some examples, the drive mechanism 80 can comprise a handle member 98 that is coupled to the rack 88 and can be used to translate the rack 88 manually. Rotation of the pinion gear 90 causes the drive shaft 82 to rotate, which in turn causes the spool 20 to rotate. Thus, the rack 88 and pinion gear 90 of the drive mechanism 80 can be operated to rotate the spool 20 within the enclosure 34 and control translation of the guide member 22 and coupled clearance member 28 within the medical tube 12 to engage and break up clots or occluding material in the medical tube 12. As the clearance member 28 engages and breaks up clots or occluding material in the medical tube 12, suction inside of the medical tube 12 from the vacuum source 62 can draw the clots or occluding material from the medical tube 12 and through the drainage tube 60 proximally towards a drainage receptacle.

In some examples, the device 14 can further comprise a drive housing 100 for the drive mechanism 80. The rack 88 of the drive mechanism 80 can be provided within and translatable through the drive housing 100. Moreover, at least a portion of the drive shaft 82, pinion gear 90 and handle member 98 can be provided within the housing 100. For example, at least a portion of the drive shaft 82 will typically be located within the enclosure 34, such that the spool 20 is journaled for rotation about that shaft. The housing 100 can comprise one or more opening portions that provide access to an interior of the housing 100. For example, the housing 100 can comprise a drive shaft opening 102 through which the drive shaft 82 can extend, and a handle member opening portion 104 through which the handle member 98 can extend. The drive housing 100 can shield the exposed portion of the drive shaft 82 and the pinion gear 90 from debris in order to prevent contamination from encountering the seal member (hereinafter described) through which the drive shaft 82 penetrates the drive housing 100 in order to help preserve the sterility within the drive housing 100.

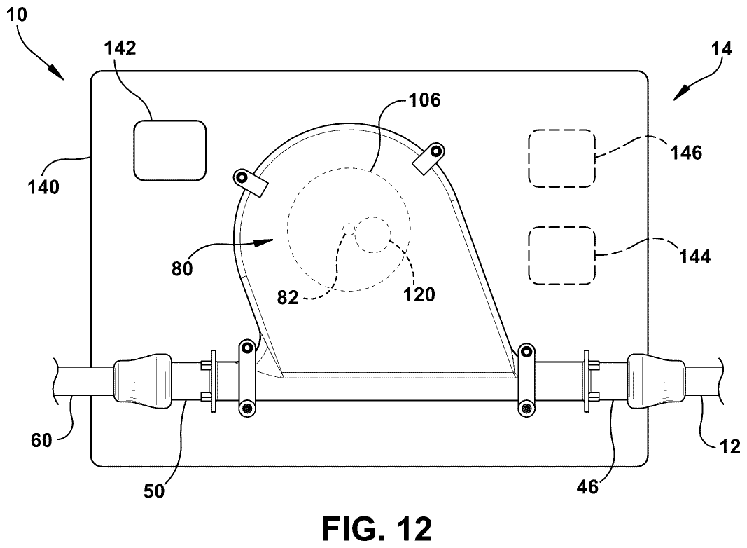

In a further embodiment, the drive mechanism 80 can comprise a motor 106 that is operable to selectively rotate the drive shaft 82 and the spool 20 coupled to the drive shaft 82, as shown in FIG. 12. In some examples, the motor 106 can be coupled directly to the drive shaft 82, thus replacing the pinion gear 90 and rack 88. In other examples, the motor 106 can drive the pinion gear 90, the rack 88 or some other transmission mechanism to selectively rotate the drive shaft 82 and the spool 20.

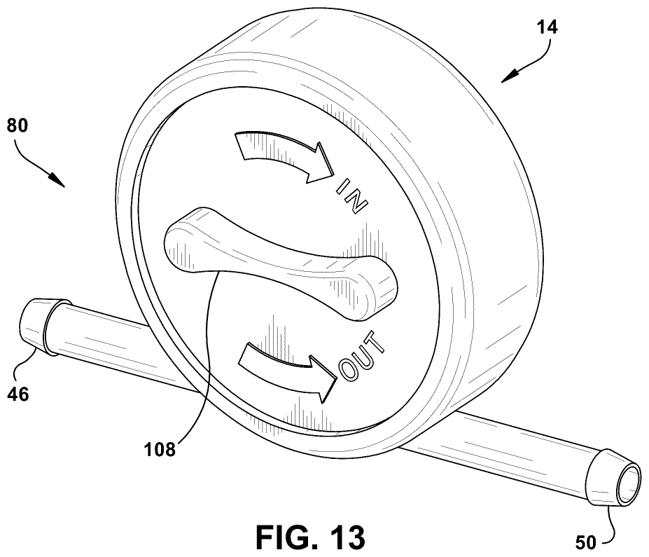

In yet a further embodiment, the drive mechanism 80 can comprise a rotatable knob 108 that can be connected to rotate the drive shaft 82 and the spool 20 coupled to the drive shaft 82, as shown in FIG. 13. The knob 108 can be coupled directly to the drive shaft 82, replacing the pinion gear 90 and rack 88. In other embodiments, the knob 108 can be rotated to drive the pinion gear 90 to rotate the drive shaft 82 and the spool 20. The knob 108 can be configured such that one complete manual rotation of the knob 108 will correspond to a pre-set number of rotations of the spool 20; i.e., so that one manual rotation of the knob 108 will result in varying degrees of insertion or retraction of the guide member 22 in the medical tube 12.

In the example embodiments of the drive mechanism 80 described above, the drive shaft 82 extends through a drive shaft opening 92 of the enclosure 34 in order to couple with external elements (e.g. within drive housing 100) for rotating the shaft and thus the spool 20. Referring now to FIGS. 10-11, to help preserve the sterile field within the enclosure 34 and prevent the introduction of contaminants through drive shaft opening 92, the device 14 can further comprise a seal member 110 configured to inhibit fluid communication between the interior 36 and the exterior 38 of the enclosure 34 through the drive shaft opening 92. For instance, in some examples the seal member 110 can comprise an O-ring that is compressed between the drive shaft 82 and the drive shaft opening 92 to form a seal therebetween, as shown in FIG. 10. In other embodiments, the seal member 110 can comprise a wiper gasket (e.g., a diaphragm seal) that also forms a seal between the drive shaft 82 and the drive shaft opening 92, as shown in FIG. 11. The wiper gasket has the ability to expand and contract in the axial direction X, which can help compensate for axial movement of the drive shaft 82. Moreover, the wiper gasket can provide a seal that provides less resistance to rotation of the drive shaft 82 but still helps to preserve the sterile field within the enclosure 34.

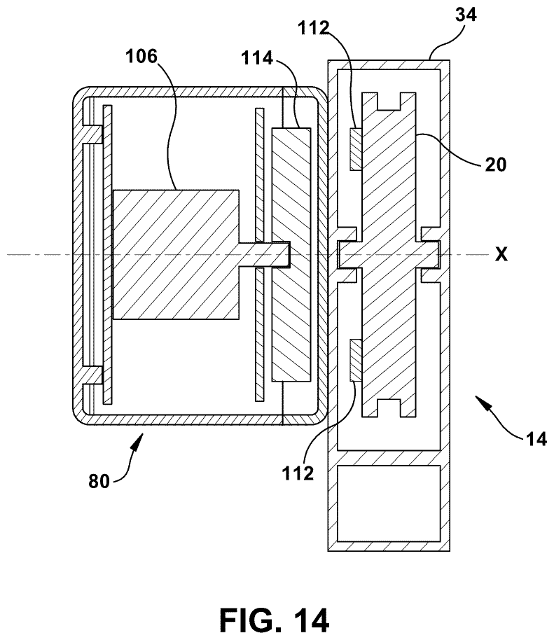

Turning now to FIG. 14, in a further embodiment the drive mechanism 80 can comprise one or more magnetic spool elements 112 that are provided within the interior 36 of the enclosure 34 and coupled to the spool 20. The drive mechanism 80 can further comprise one or more magnetic drive elements 114 that are provided outside of the enclosure 34 and magnetically coupled to the one or more spool elements 112. For instance, each spool element 112 can comprise a magnet or a magnetic material that can be magnetically coupled to one or more drive elements 114 and vice versa. The magnet can comprise various shapes and sizes (e.g., round, square, bar, etc.) and can have various magnetization orientations (e.g., axial, diametric, etc.).

The drive elements 114 can be magnetically coupled to the spool elements 112 such that revolution of the drive elements 114 about the axis X causes the spool elements 112 and thereby spool 20 to rotate about that axis. To rotate the drive elements 114, the drive elements 114 can be coupled to a motor 106, which can be operable to selectively rotate the drive elements 114. In an alternative example (not shown) the drive elements 114 can be coupled to a handle or crank that can be reversibly mated to the enclosure 34 when it is desired to rotate the spool 20 within in order to actuate the clearance member. In this embodiment, the associated handle or crank can be manually rotated once the drive elements 114 have been magnetically coupled through the enclosure 34 wall to the associated spool elements 112, thereby driving the spool and actuating the clearance member.

The drive elements 114 and spool elements 112 in the embodiment described above may be provided on opposite sides of a wall of the enclosure 34 and can be magnetically coupled through the wall of the enclosure 34. Thus, in the above embodiment there is no need for a drive shaft to penetrate through an opening in the enclosure 34. As such, the magnetic actuation of the spool 20 described in the above embodiment can eliminate the need for a drive-shaft opening, thus eliminating a potential pathway for contamination of the sterile field within the enclosure 34.

In any of the example embodiments of the drive mechanism 80 described above, the drive mechanism 80 can comprise a transmission mechanism 120 that is coupled between a driving element (e.g. motor, pinion gear, rotating handle, crank, etc.) and a driven element (e.g. the drive shaft of the spool). The transmission mechanism transmits driving force from the driving element to the driven element. For example, as schematically shown in FIG. 12, the transmission mechanism 120 may be coupled to and configured to transmit rotational force between the motor 106 and the drive shaft 82. In other examples, the transmission mechanism 120 may be coupled to and configured to transmit rotational force to the drive shaft 82 from a pinion gear 90, knob 108, or any other element of the drive mechanism 80 that is movable to cause rotation of the drive shaft 82. The transmission mechanism 120 can incorporate one or a series of gears that effectively fix or adjust a drive ratio between the driving element and the driven element as known in the art. For instance, the transmission mechanism 120 can adjust or fix the drive ratio such that one complete rotation of the driving element (e.g. handle or motor crank) produces two, three, four, or any other number of corresponding rotations of the spool 20 to either wind or unwind the guide member 22. The transmission mechanism 120, if present, can be used to control the degree of rotation of the spool 20, and correspondingly how far the guide member 22 is advanced or retracted, based on the degree of rotation (e.g. manual actuation) of the driven member, such as a handle rotated by hand or a pinion gear driven by a rack 88.

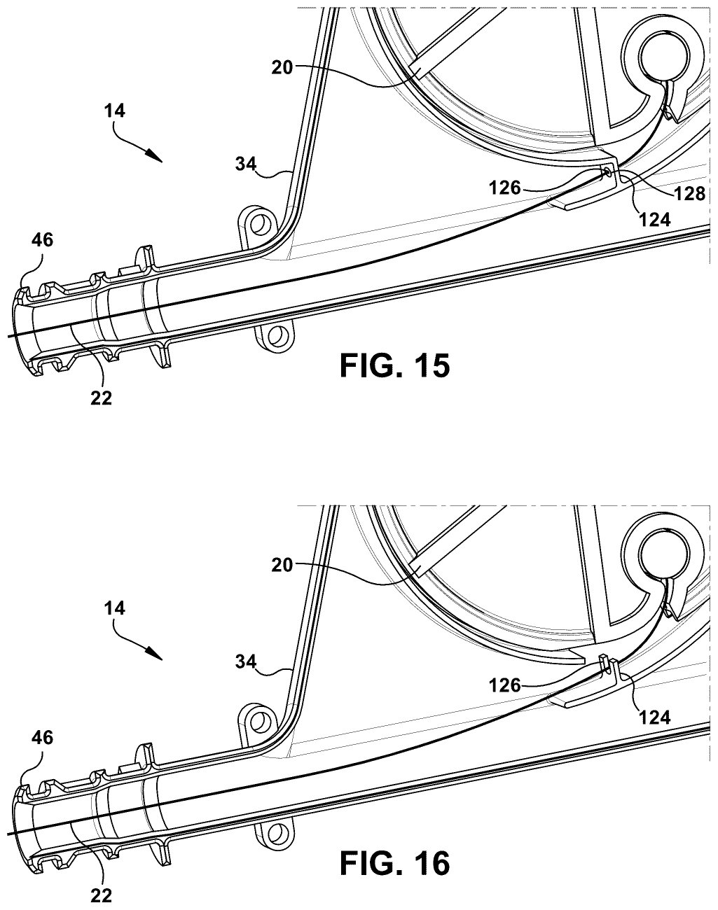

Turning now to FIGS. 15 & 16, in some embodiments the device 14 can further comprise a guide portion 124 that can be configured to help direct the guide member 22 onto or off of the spool 20. More specifically, the guide portion 124 can comprise a guide channel 126 that the guide member 22 translates through when the spool 20 is rotated. The guide channel 126 can be an aperture that extends through the guide portion 124 (as shown in FIG. 15) or the guide channel 126 can be notch or slot that extends inward from an edge of the guide portion 124 (as shown in FIG. 16). In some examples, the guide portion 124 can comprise a gasket 128 that defines the guide channel 126, as shown in FIG. 15. The gasket 128 can comprise a rubber material or any other material that can provide a smooth surface for the guide member 22 to rub against when translating through the guide channel 126. Moreover, the gasket 128 can be configured to provide a seal that inhibits the transfer of fluid or other materials through the guide channel 126 and into the area surrounding the spool 20.

The guide channel 126 can comprise a dimension that is equal to or slightly larger than a dimension of the guide member 22. For example, if the guide channel 126 is an aperture, the diameter of the aperture may be equal to or slightly larger than a diameter of the guide member 22. As another example, if the guide channel 126 is an open slot, the width of the open slot may be equal to or slightly larger than a width of the guide member 22. By “slightly larger” it is meant that the difference between the dimension of the guide channel 126 and the dimension of the guide member 22 is preferably less than or equal to 0.005 inches and still more preferably, less than or equal to 0.001 inches. When the guide channel 126 comprises a dimension that is equal to or slightly larger than a dimension of the guide member 22, the guide portion 124 can help scrape debris or other material off of the guide member 22 as the guide member 22 translates through the guide channel 126. However, the guide channel 126 can be more than slightly larger in dimension than the guide member 22 in some embodiments and in some embodiments, the guide channel 126 can be smaller in dimension than a the guide member 22 to produce an interference fit.

In some examples, the guide portion 124 can comprise a one-way elastomeric valve or brush that can be configured to scrape off the guide member 22 as the guide member 22 is wound onto or off of the spool 20. The guide portion 124 can comprise any portion that is configured to direct and/or scrape the guide member 22 as the guide member 22 is wound onto or off of the spool 20.

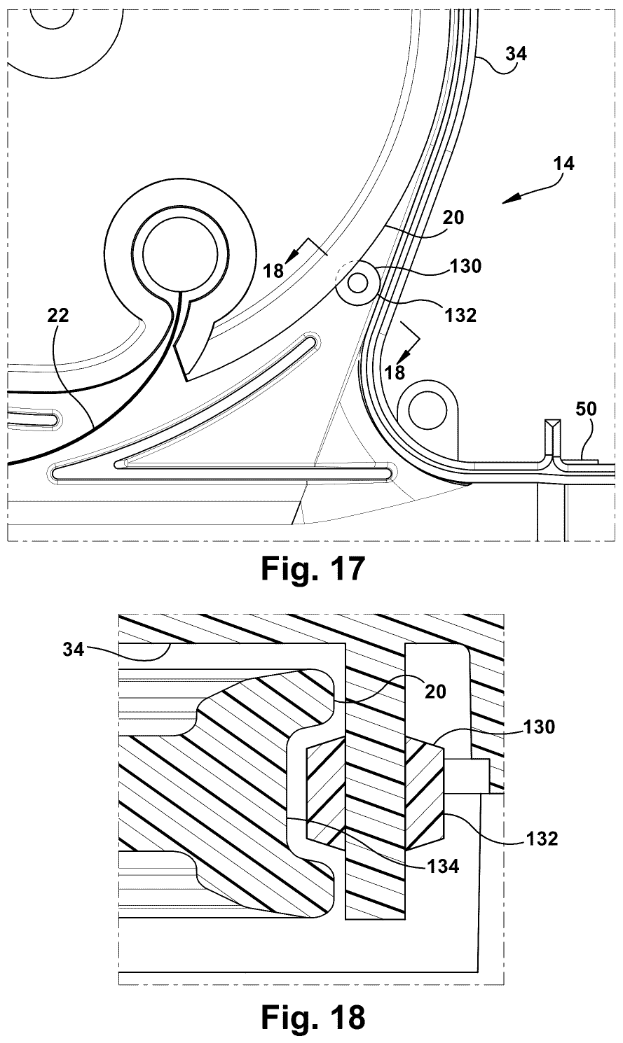

In some embodiments, the device 14 can further comprise a compression element 130 that presses the guide member 22 against the spool 20 while the guide member 22 is wound about the spool 20, as shown in FIGS. 17-20. The compression element 130 can comprise one or more compression wheels 132 (as shown in FIGS. 17 & 18). The compression wheels 132 may be provided about the circumference of the spool 20 and coupled to the enclosure 34. The compression wheels 132 can be fixed within the enclosure 34 or the compression wheels 132 can be rotatable about an axis parallel to the axis X. In some examples, the spool 20 can comprise a circumferential groove 134 and the compression wheels 132 can extend at least partially within the circumferential groove 134.

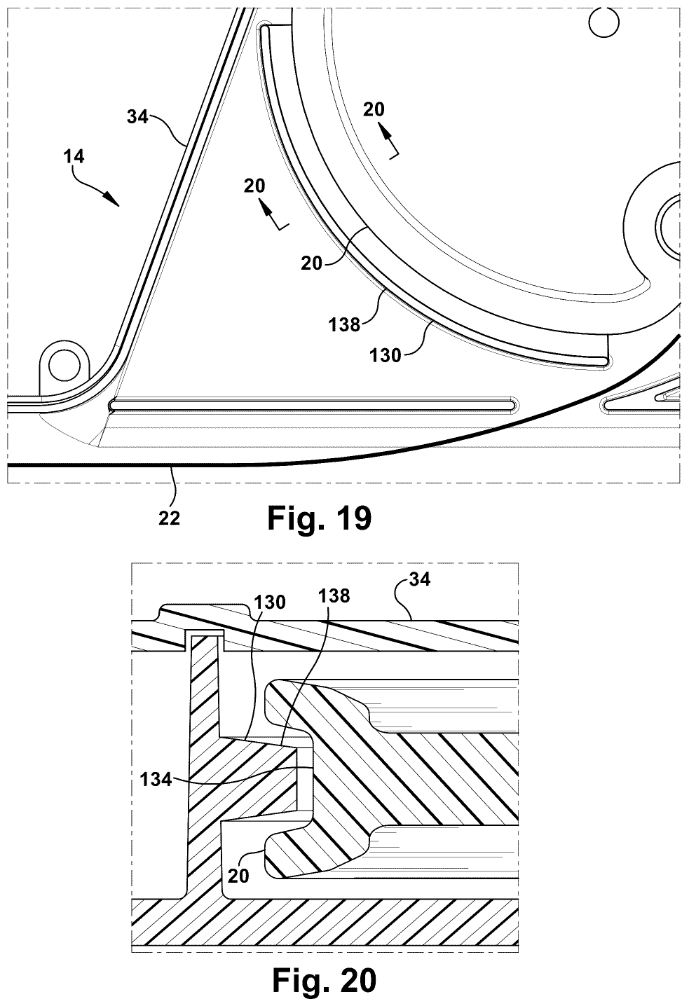

The compression element 130 can additionally or alternatively comprise at least one rib 138 (as shown in FIGS. 19 & 20). The rib 138 can extend about at least a portion of the circumference of the spool 20. For examples wherein the spool 20 comprises the circumferential groove 134, the rib 138 can extend at least partially within the circumferential groove 134.

The compression wheels 132 and/or rib 138 of the compression element 130 can be distanced from the spool 20 such that a gap is provided between the spool 20 and the compression wheels 132 and/or rib 138 for the guide member 22 to extend through when wound about the spool 20. The gap can be sized such that the compression wheels 132 and/or rib 138 of the compression element 130 will press the guide member 22 against the spool 20 while the guide member 22 is wound about the spool 20, thus helping to ensure that the guide member 22 will be wound tightly against the spool 20 and inhibiting the guide member 22 from binding as the guide member 22 is wound onto or off of the spool 20.

In some embodiments, the device 14 can comprise a control system 140 configured to automatically control operation of the drive mechanism 80 and/or detect various operating parameters of the medical tube assembly 10, as shown schematically in FIG. 12.

For example, in some embodiments, the control system 140 can be configured to selectively operate the motor 106 to rotate the spool 20 and thus stroke the guide member 22 to provide one or more predetermined stroke cycles. One stroke can be an actuation of the guide member 22 from an advanced state to a retracted state and back to the advanced state. Alternatively, one stroke can be an actuation of the guide member 22 from a retracted state to an advanced state and back to the retracted state. In a further alternative, one stroke can refer to actuation of the guide member 22 only between the retracted and advanced states or vice versa. The precise scope of actuation of the guide member 22 constituting a ‘stroke’ in a particular case may be determined in the judgment of the clinicians responsible for patient care.

The control system 140 can be configured to stroke the guide member 22 to provide a predetermined stroke cycle wherein the guide member 22 is stroked intermittently for a set number of strokes with a set period of time between and/or during strokes. As another example, the control system 140 can be configured to stroke the guide member 22 to provide a predetermined stroke cycle wherein the guide member 22 is stroked continuously for a set number of strokes with no period of time between strokes. As another example, the control system 140 can be configured to stroke the guide member 22 to provide a predetermined stroke cycle wherein the guide member 22 is stroked only once. The control system 140 can be configured to stroke the guide member 22 to provide a plurality of different predetermined stroke cycles.

The control system 140 can comprise a user interface 142 that can permit the user to select and/or initiate execution of a predetermined stroke cycle and/or adjust variables of the stroke cycle such as how many times the guide member 22 should be stroked, how much time is between strokes, a position of the guide member 22 in the retracted state, a position of the guide member 22 in the advanced state, or any other variable. The user interface 142 can comprise a touch-screen, one or more switches or buttons, or any other feature that permits a user to select and/or initiate execution of the predetermined stroke cycle and/or adjust variables of the stroke cycle.

In some embodiments, the control system 140 can comprise a sensor 144 configured to detect one or more operating parameters of the medical tube assembly 10. For example, in one embodiment the sensor 144 can be configured to detect at least one or more of the following operating parameters: a) a degree of translation of the guide member 22 and/or clearance member 28; b) a degree of rotation of the spool 20; c) a length and/or position of the distal opening 18 of the medical tube 12; d) a torque in the drive mechanism 80 such as, for example, a torque in the drive shaft 82, pinion gear 90, or the axle portion 96 of the spool 20; e) a pressure in the medical tube 12; f) a pressure in the drainage tube 60; and g) any other operating parameter of the medical tube assembly 10. For instance, the sensor 144 may be a continuity circuit used to detect a position of a break/or cut in the medical tube 12, a hall effect sensor, a torque sensor, a pressure sensor, or some other type of sensor configured to detect one or more operating parameters of the medical tube assembly 10.

The control system 140 can be configured to selectively operate the motor 106 based on the operating parameter detected by the sensor 144. For instance, if the sensor 144 detects a pressure in the medical tube 12 or drainage tube 60 that indicates the presence of an obstruction in the medical tube 12, the control system 140 can be configured to operate the motor 106 to stroke the guide member 22 and coupled clearance member 28 through the medical tube 12 and help dislodge and/or draw the obstructing material within the medical tube 12. As another example, if the sensor 144 detects a torque in the drive mechanism 80 during operation that exceeds an upper limit set for the motor 106 or some other component of the drive mechanism 80, the control system 140 can be configured to stop operation of the motor 106 and optionally sound an alarm.

In some embodiments, the operating parameter detected by the sensor 144 may be defined to have a lower limit and/or upper limit for each variable. Moreover, the control system 140 can comprise an alarm 146 that is configured to selectively activate based on the operating parameter detected by the sensor 144. For example, the alarm 146 can be configured to activate when the detected operating parameter is at or below the lower limit or at or above the upper limit. For instance, the alarm 146 can be configured to activate when a detected torque in the drive mechanism 80 exceeds an upper limit for the motor 106 or some other component of the drive mechanism 80.

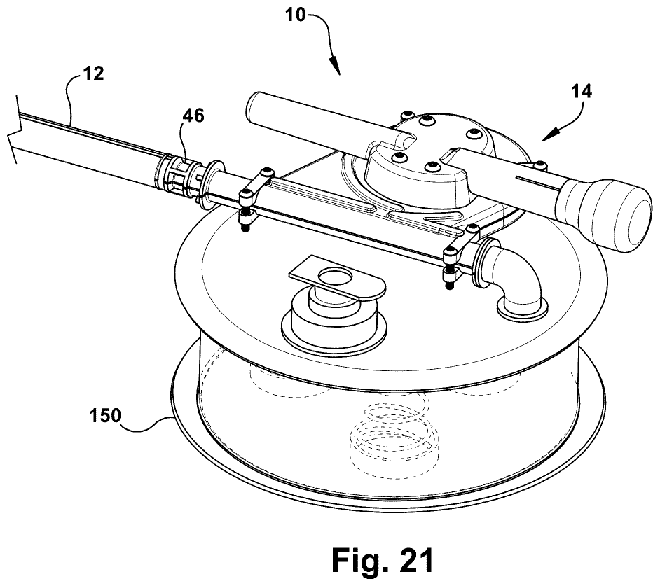

The medical tube assembly 10 can comprise a drainage receptacle 150 into which material passing through the device 14 and/or drainage tube 60 can be delivered, as shown in FIG. 21. FIG. 21 shows an onboard drainage receptacle that is portable and coupled to the clearance device 14, however other configurations are possible. In the illustrated embodiment the device 14 is coupled to the receptacle 150 and oriented such that the medical tube connected thereto extends at an angle (e.g. a 90° angle) relative to the pathway through a port in the receptacle 150 through which evacuated obstructing material will be deposited into the receptacle 150. In this embodiment such material will negotiate a bend or turn in the vacuum pathway (as shown in FIG. 21). Alternatively, the device 14 can be oriented such that its connection to the medical tube 12 results in a substantially linear path from the medical tube 12 into the receptacle 150 through the aforementioned port so that evacuated obstructing material will be deposited into the canister 150 along a substantially linear vacuum pathway and the elbow shown in FIG. 21 (or other path-redirecting structure) will not be necessary.

The receptacle 150 can take on a variety of different configurations without departing from the scope of the invention. For example, the receptacle 150 can be a spring loaded drainage canister, a bulb drain canister, a chest drainage canister, or any other type of drainage receptacle into which material passing through the device 14 and/or drainage tube 60 can be delivered.

As described above, the device 14 of the medical tube assembly 10 can be coupled to the medical tube 12 and the drive mechanism 80 of the device 14 can be operated to rotate the spool 20 and actuate the guide member 22 and coupled clearance member 28 through the medical tube 12 to help dislodge and/or draw the obstructing material within the medical tube 12. In some embodiments, the device 14 can comprise additional means to assist in actuation of the guide member 22 such as, for example, ultrasonic vibration or a magnetic shuttle coupled to the medical tube 12. Moreover, in some embodiments, one or more features of the medical tube assembly 10 can be configured to help control how far the clearance member 28 translates through the medical tube 12 during actuation of the guide member 22.

For example, in some embodiments, the device 14 can be operable to move the clearance member 28 between a retracted state and a fully advanced state and one or more features of the device 14 can be calibrated to the medical tube 12 such that when the device 14 is coupled with the medical tube 12 and the clearance member 28 is in the fully advanced state, the clearance member 28 will be located at a predetermined location L1 within the medical tube 12 (see FIG. 1). As discussed in further detail below, the predetermined location L1 can be a location relative to an end of the medical tube 12 or some other portion of the medical tube 12.

For instance, in one example, the spool 20 of the device 14 can be manually or automatically rotated from a first position wherein the guide member 22 is at least partially wound about the spool 20 and the clearance member 28 is in a retracted state to a second position, wherein the guide member 22 is fully unwound from the spool 20 and the clearance member 28 is in the fully advanced state. The length of the guide member 22 can be calibrated such that when the device 14 is coupled with the medical tube 12 and the clearance member 28 is in the fully advanced state, the clearance member 28 will be located at the predetermined location L1 within the medical tube 12.

In another example, the control system 140 can be configured to selectively operate the motor 106 to rotate the spool 20 and stroke the guide member 22 such that the clearance member 28 moves between a fully advanced state and a retracted state, wherein when the device 14 is coupled with the medical tube 12 and the clearance member 28 is in the fully advanced state, the clearance member 28 will be located at the predetermined location L1 within the medical tube 12. For instance, in some examples the sensor 144 of the control system 140 can be configured to detect a length and/or position of the distal opening 18 of the medical tube 12 by using, for example, a hall effect sensor, a continuity circuit used to detect a position of a break and/or cut in the medical tube 12, or some other means. Based on the detected measurement, the control system 140 can provide a predetermined stroke cycle for the guide member 22 to stroke the guide member 22 such that when the clearance member 28 is in a fully advanced state, the clearance member 28 will be located at the predetermined location L1 within the medical tube 12. In other examples, the user interface 142 of the control system 140 can be used to select or set a predetermined stroke cycle for the guide member 22 to stroke the guide member 22 such that the clearance member 28 moves between a fully advanced state and a retracted state, wherein when the clearance member 28 is in the fully advanced state, the clearance member 28 will be located at the predetermined location L1 within the medical tube 12. The control system 140 can be configured in a variety of ways to stroke the guide member 22 such that the clearance member 28 moves between a fully advanced state and a retracted state, wherein when the clearance member 28 is in the fully advanced state, the clearance member 28 will be located at the predetermined location L1 within the medical tube 12.

The predetermined location L1 can be a location relative to the distal opening 18 of the medical tube 12 that is located within the medical tube 12 such that the clearance member 28 is preferably within 2 cm of the distal opening 18 and more preferably, within 1 cm of the distal opening 18 at the predetermined location L1. This can help ensure that the clearance member 28 passes through a substantial portion of the medical tube 12 when moving between its fully advanced state to its retracted state to help dislodge and/or draw the obstructing material within the medical tube 12. Still more preferably, the predetermined location L1 can be located within the medical tube 12 such that the clearance member 28 is spaced a distance from the distal opening 18 that is equal to or greater than 0.5 cm. This can help ensure that the clearance member 28 will not extend through the distal opening 18 of the medical tube 12 during actuation of the clearance member 28. However, in other embodiments, the predetermined location L1 can be a location relative to the proximal opening 16 that is located such that the clearance member 28 is a certain predetermined distance from the proximal opening 16 of the medical tube 12. Moreover, in some embodiments, the predetermined location L1 can be a location relative to other structure of the medical tube 12 such as, for example an aperture of the medical tube 12. The predetermined location L1 can be located such that the clearance member 28 is distal or proximal of an aperture in the medical tube 12 in the fully advanced position. The predetermined location L1 can be a location located anywhere and relative to any structure within the medical tube 12 without departing from the scope of the invention.

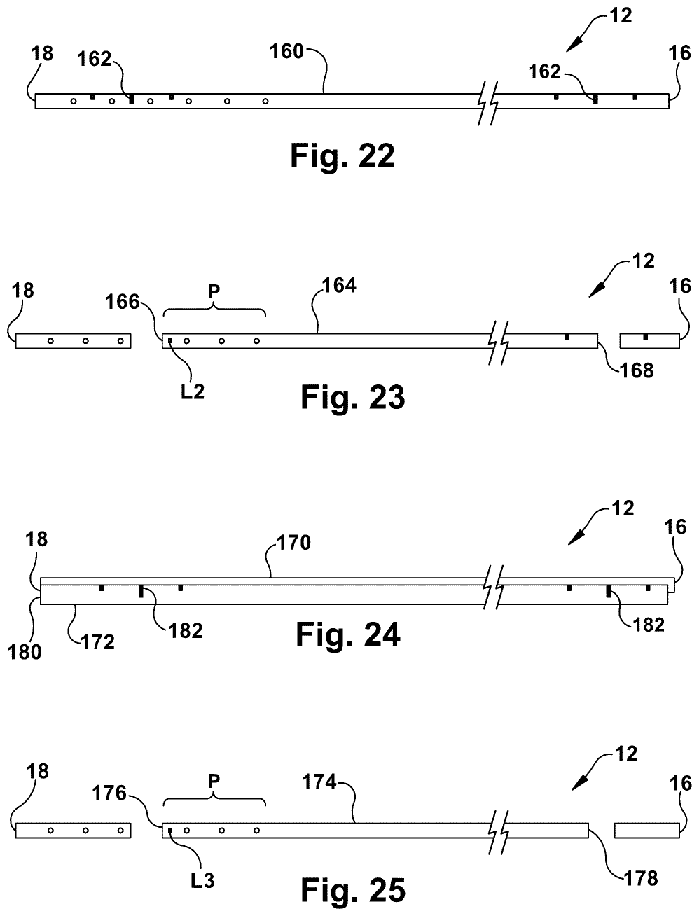

In other embodiments, the length of the medical tube 12 can be calibrated to the device 14 and a patient. More specifically, the length of the medical tube 12 can be adjusted such that when the device 14 is coupled with the length adjusted medical tube and the clearance member 28 is in the fully advanced state, the clearance member 28 will be located at a predetermined or user-selected location within the adjusted medical tube. Moreover, the length of the medical tube 12 can be adjusted such that a distal, residing portion P of the adjusted medical tube will be correctly sized for a patient. For instance, in one example the medical tube 12 can comprise a main body 160 and at least one pair of markings 162 that is arranged on the main body 160 such that cutting the main body 160 at each marking 162 will create a cut tube portion 164 with a distal end 166 and a proximal end 168, as shown in FIGS. 22 & 23. Each marking 162 can be a drawn line, a notch, a projection, or any other feature that can indicate a position to cut the main body 160. Moreover, a plurality of such markings 162 can be provided as graduations along a line or multiple lines that are aligned with a longitudinal axis of the medical tube 12 in order to facilitate a desired residing-portion P length (which will reside inside a patient in use) while ensuring that the overall length of the medical tube 12 is calibrated to accommodate the clearance member 28 in its fully-advanced state.

By cutting the main body 160 at a selected marking 162 near its distal end, the cut tube portion 164 will have a distal, residing portion P (e.g. wherein apertures for the drainage of fluid are provided in the tube 12) having a predetermined or user-selected length. The residing portion P can be a portion of the cut tube portion 164 that is intended to reside entirely within a patient when the distal end 166 of the cut tube portion 164 is located in a desired compartment of a patient. For instance, the residing portion P can be defined by a section of the cut tube portion P having a branched lumen or a plurality of apertures (noted above) that is desired to reside entirely within the patient. By cutting the main body 160 at a selected marking 162 near its distal end, the residing portion P can have a predetermined or user-selected length matched to a patient such that when the distal end 166 of the cut tube portion 164 is located in a desired compartment of the patient, the residing portion P will reside entirely within the patient and will not extend outside of the patient.

By cutting the main body 160 at the marking 162 near its proximal end that corresponds to the marking 162 where the tube was cut near its distal end (to define the length of the residing portion P), the cut tube portion 164 will be configured such that when the device 14 is coupled with the cut tube portion 164 and the clearance member 28 is in the fully advanced state, the clearance member 28 will be located at a predetermined or user-selected location L2 within the cut tube portion 164. The location L2 can be a location relative to the distal end 166 that is located within the cut tube portion 164 such that the clearance member 28 is preferably within 2 cm of the distal end 166 and more preferably, within 1 cm of the distal end 166 at the location L2. Still more preferably, the location L2 can be a location within the cut tube portion 164 such that the clearance member 28 is spaced a distance from the distal end 166 that is equal to or greater than 0.5 cm. However, the location L2 can be any predetermined or user-selected location relative to any structure of the cut tube portion 164 without departing from the scope of the invention.

In the example described above, the at least one pair of markings 162 is configured for calibration of the medical tube 12 for use with the device 14 described above and to produce a residing portion P having a particular, predetermined or user-selected length. However, in some embodiments, the at least one pair of markings 162 can similarly be configured for calibration of the medical tube 12 for use with other devices that are operable to move a clearance member between a fully advanced state and a retracted state. Furthermore, the length of the produced residing portion P can vary in different embodiments. Still further, in some embodiments the medical tube 12 can comprise multiple pairs of associated markings, wherein each pair of associated markings is configured to produce an residing portion P having a particular length and to calibrate the medical tube 12 for use with a particular device that is operable to move a clearance member between a fully advanced state and a retracted state. In this manner, the medical tube 12 can be calibrated using the pairs of markings for use with various clearance devices and to produce residing portions P of various lengths that are matched to a particular patient/procedure. If multiple pairs of markings are provided, each pair can be distinguished using different numbers, letters, colors, marking lengths, or any other means to distinguish between the multiple pairs of markings.

In another example, the medical tube 12 can comprise a main body 170 and the medical tube assembly 10 can comprise an indicator device 172 that is configured to be aligned with the medical tube 12 such that alignment with the medical tube 12 will indicate a pair or respective pairs of locations to cut the main body 170 at to create a cut tube portion 174 having a calibrated length with a distal end 176 and a proximal end 178, as shown in FIGS. 24 & 25. For instance, in one example, the indicator device 172 can be a guide that comprises an end 180 and at least one pair of indicators 182. The indicator device 172 can be aligned longitudinally with the medical tube 12 such that the end 180 is aligned with the proximal opening 16 or distal opening 18 of the medical tube 12. When aligned as such, the pair of indicators 182 can indicate a pair of locations to cut the main body 170 to create the cut tube portion 174. Each indicator 182 can be a line, a notch, a projection, an aperture, or any other feature that can indicate a location along its length. In other examples, the indicator device 172 can be a sleeve that the medical tube 12 can be inserted into, wherein when the medical tube 12 is inserted within, a pair of indicators of the sleeve can indicate a pair of locations to cut the main body 170 to create the cut tube portion 174. The indicator device 172 can be any device configured to be aligned with the medical tube 12 such that alignment with the medical tube 12 will indicate a pair or pairs of locations to cut the main body 170 to create a cut tube portion 174 having a calibrated length while defining a desired-length residing portion P.

By cutting the main body 170 at an indicated location near its distal end, the cut tube portion 174 will have a distal, residing portion P having a predetermined or user-selected length as described above. The residing portion P can be a portion of the cut tube portion 174 that is intended to reside entirely within a patient as described above. For instance, the residing portion P can be defined by a section of the cut tube portion P having a branched lumen or a plurality of apertures that is desired to reside entirely within the patient. By cutting the main body 170 at a particular indicated location near its distal end, the residing portion P can have a predetermined or user-selected length matched to a patient such that when the distal end 176 of the cut tube portion 174 is located in a desired compartment of the patient, the residing portion P will reside entirely within the patient and will not extend outside of the patient.

By cutting the main body 170 at the indicated location near its proximal end that is associated with or corresponds to the indicated location where it was cut near its distal end, the cut tube portion 174 will be configured such that when the device 14 is coupled with the cut tube portion 174 and the clearance member 28 is in the fully advanced state, the clearance member 28 will be located at a predetermined or user-selected location L3 within the cut tube portion 174. The location L3 can be a location relative to the distal end 176 that is located within the cut tube portion 174 such that the clearance member 28 is preferably within 2 cm of the distal end 176 and more preferably, within 1 cm of the distal end 176 at the location L3. Still more preferably, the location L3 can be a location within the cut tube portion 174 such that the clearance member 28 is spaced a distance from the distal end 176 that is equal to or greater than 0.5 cm. However, the location L3 can be any location relative to any structure of the cut tube portion 164 without departing from the scope of the invention.

In the example described above, the at least one pair of indicators 182 is configured for calibration of the medical tube 12 for use with the device 14 described above and to produce a residing portion P having a particular, predetermined or user-selected length. However, in some embodiments the at least one pair of indicators 182 can be similarly configured for calibration of the medical tube 12 for use with other devices that are operable to move a clearance member between a fully advanced state and a retracted state. Furthermore, the predetermined or user-selected length of the residing portion P can vary in different embodiments. Still further, in some embodiments the indicator device 172 can comprise multiple pairs of indicators, wherein each pair of indicators is configured to produce a residing portion P having a particular, predetermined or user-selected length and to calibrate the medical tube 12 for use with a particular device that is operable to move a clearance member between a fully advanced state and a retracted state. In this manner, the medical tube 12 can be calibrated using the indicator device 172 to be used with various clearance devices and to produce residing portions P of various lengths matched to a particular patient/procedure. If multiple pairs of indicators are provided, each pair can be distinguished using different numbers, letters, colors, indicator portion lengths, or any other means to distinguish between the multiple pairs of indicator portions.

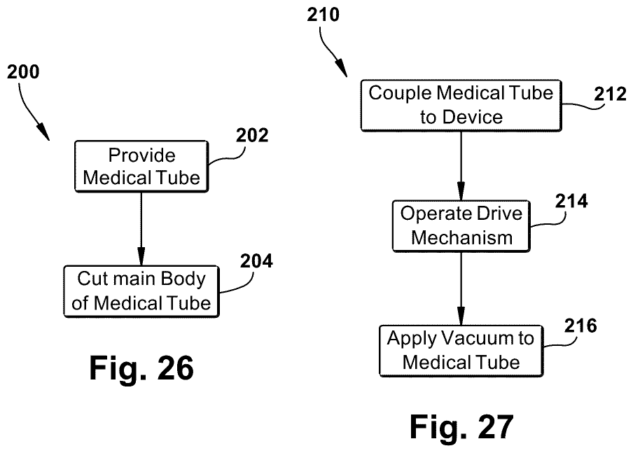

An example method 200 will now be described of calibrating a medical tube for a clearance device that is operable to move a clearance member of the clearance device between a fully advanced state and a retracted state. The clearance device may be the device 14 described above or the clearance device may be some other clearance device. As shown in FIG. 26, the method 200 can comprise the step 202 of providing a medical tube comprising a main body such as, for example, the medical tube 12 comprising the main body 160 described above. The method 200 can further comprise the step 204 of cutting the main body to create a cut tube portion with a distal end and a proximal end, the cut tube portion being configured such that when the cut tube portion is coupled with the clearance device and the clearance member is within the cut tube portion in the fully advanced state, the clearance member will be located at a predetermined location within the cut tube portion. The produced cut tube portion can also have a distal, residing portion comprising a predetermined length such that when a distal end of the cut tube portion is located in a desired compartment of a patient, the residing portion will be located entirely within the patient such that the residing portion P will not extend outside of the patient. For example, the medical tube 12 can comprise the at least one pair of markings 162 described above and the step 204 can comprise the step of cutting the main body 160 at the markings 162 to create the cut tube portion. As another example, the step 204 can comprise the steps of a) providing an indicator device comprising an indicator portion such as, for example, the indicator device 172 comprising the at least one pair of indicator portions 182 described above; b) aligning the indicator device with the medical tube such as, for example, by aligning the end 180 of the indicator device 172 the proximal opening 16 or distal opening 18 of the medical tube 12; and c) cutting the main body at a location indicated by the pair of indicator portions to create the cut tube portion.

As shown in FIG. 27, in a further method 210 a step 212 of coupling the medical tube 12 to the distal opening 42 of the device 14 is performed, for example by using either of connectors 52, 66 as described above. The method 210 can further comprise the step 214 of operating the drive mechanism 80 to rotate the spool 20 and thereby move the guide member 20 and coupled clearance member 28 within the medical tube 12 between an advanced state and a retracted state without compromising a sterile field within the enclosure 34. For example, the drive mechanism 80 can be operated using the rack 88 and pinion gear 90, the motor 106, the knob 108, the one or more drive elements 114, and/or the control system 140 as discussed above. The method 210 can further comprise the step 216 of applying a vacuum to the medical tube 12 while the guide member 20 and coupled clearance member 28 are moved between the advanced state and retracted state. This can be accomplished for example by coupling one end of the drainage tube 60 to the vacuum source 62 and another end of the drainage tube 60 to either the proximal opening 50 of the device 14 or the 3-way connecter 66 and then operating the vacuum source 62 to apply the vacuum.

Illustrative embodiments have been described, hereinabove. It will be apparent to those skilled in the art that various modifications and variations can be made without departing from the spirit and scope of the claimed invention. It is intended to include all such modifications and alterations within the scope of the present invention.

What is claimed is:

1. A medical device for clearing obstructions from a medical tube, the medical device comprising:
an enclosure defining:
(i) an interior comprising a sterile field, a chamber, and a discrete conduit in communication with and extending substantially tangentially of said chamber,
(ii) an exterior, and
(iii) an opening for providing access to the interior;
a drive mechanism; and
an elongated guide member,
the drive mechanism being configured to (i) advance the elongated guide member out of the enclosure through the opening without compromising the sterile field and (ii) withdraw the elongated guide member into the enclosure through the opening without compromising the sterile field.

2. The medical device of claim 1, wherein the drive mechanism comprises a drive shaft extending through a drive shaft opening of the enclosure, wherein rotation of the drive shaft is configured to cause advancement of the elongated guide member out of the enclosure or withdrawal of the elongated guide member into the enclosure.

3. The medical device of claim 2, further comprising a seal member configured to inhibit fluid communication between the interior of the enclosure and the exterior of the enclosure through the drive shaft opening.

4. The medical device of claim 3, wherein the seal member comprises an O-ring or a wiper gasket and provides a seal between the drive shaft and the drive shaft opening.

5. The medical device of claim 1, further comprising a medical tube connector that is coupled to the opening of the enclosure and removably couplable to a medical tube to form a closed passageway for fluid communication between the discrete conduit and the medical tube through the medical tube connector.

6. The medical device of claim 1, further comprising a drainage tube connector that is coupled to the discrete conduit and removably couplable to a drainage tube.

7. The medical device of claim 6, wherein a vacuum source is coupled to the drainage tube to provide a vacuum to draw fluid into the drainage tube.

8. The medical device of claim 1, further comprising a clearance member coupled to a distal end of the elongated guide member.

9. The medical device of claim 1, further comprising a control system configured to automatically control operation of the drive mechanism.

10. The medical device of claim 9, wherein the control system comprises a sensor configured to detect an operating parameter.

11. The medical device of claim 10, wherein the operating parameter is a degree of translation of the elongated guide member.

12. The medical device of claim 10, wherein the operating parameter is a length of a medical tube that is coupled to the discrete conduit.

13. The medical device of claim 10, wherein the operating parameter is a torque in the drive mechanism.

14. The medical device of claim 10, wherein the operating parameter is a pressure in a medical tube that is coupled to the discrete conduit.

15. The medical device of claim 10, wherein the operating parameter is a pressure in a drainage tube that is coupled to the discrete conduit.

16. The medical device of claim 10, further comprising an alarm configured to activate based on the operating parameter detected by the sensor.

17. The medical device of claim 10, wherein the control system is further configured to automatically control operation of the drive mechanism based on the operating parameter detected by the sensor.

18. The medical device of claim 1, wherein said discrete conduit extends linearly between opposite first and second ends, said opening being provided at said first end.

19. The medical device of claim 18, further comprising a medical tube connector and a drainage tube connector, wherein said medical tube connector is coupled to the first end and removably couplable to a medical tube to form a closed passageway for fluid communication between the discrete conduit and the medical tube through the medical tube connector, and wherein the drainage tube connector is coupled to the second end and removably couplable to a drainage tube.

20. A medical tube assembly, comprising:
the medical device of claim 1;
a medical tube; and
a drainage receptacle,
wherein said discrete conduit extends between opposite first and second ends, said opening being provided at said first end, wherein the first end of the discrete conduit is removably coupled to the medical tube, and wherein the second end of the discrete conduit is removably coupled to the drainage receptacle such that the medical device is fluidly disposed between the medical device and the drainage receptacle.
